(19) United States
(12) Patent Application Publication
(10) Pub. No.: US 2005/0112635 A1
(43) Pub. Date: May 26, 2005

(54) Quantification of analytes using internal standards

(75) Inventors: Thomas Gentle, Red Lion, PA (US); Richard Moore, Glenville, PA (US); Thomas Winegar, Hawthorne, NJ (US); Song Shi, Reisterstown, MD (US); Zhe Jin, Cockeysville, MD (US)

Correspondence Address: DAVID W HIGHET VP AND CHIEF IP COUNSEL;BECTON DICKINSON AND COMPANY, 1 BECTON DRIVE, MC110, FRANKLIN LAKES, NJ, 07417-1880, US

(73) Assignee: Becton, Dickinson and Company, Franklin Lakes, NJ (US)

(21) Appl. No.: 10/945,891

(22) Filed: Sep. 22, 2004

Related U.S. Application Data
(60) Provisional application No. 60/504,429, filed on Sep. 22, 2003.

(51) Int. Cl. C12Q 1/68; G01N 33/53; G01N 33/00

(52) U.S. Cl. 435/6; 435/7.1; 436/86; 436/94

(57) ABSTRACT

The present invention pertains to methods of quantifying the levels of at least one analyte in a sample or extract comprising adding a known quantity of at least one internal standard to the sample or extract. The present invention also relates to internal standards used in mass spectrometry, as well as compositions thereof. Internal standards for mass spectrometry according to the invention can be used, for example, to assist aligning mass spectra obtained from two different samples, each of which comprises the internal standard. In one aspect of the invention, the internal standard is a dendrimer. A labile internal standard may be used in conjunction with the dendrimer.

### FIELD OF THE INVENTION

The present invention pertains to methods of quantifying the levels of at least one analyte in a sample or extract comprising adding a known quantity of at least one internal standard to the sample or extract using mass spectrometry. The present invention also relates to internal standards used in mass spectrometry, as well as compositions thereof. The present invention further relates to the use of at least one internal standard in a specimen collection device.

### BACKGROUND OF THE INVENTION

Applicants make no admission that any of the following cited articles and methods are prior art, and they expressly reserve the right to demonstrate, where appropriate, that these articles and methods do not constitute prior art under the applicable statutory provisions.

With the near completion of the human genome mapping, scientists have turned their attention to gene products. As the phenotypic products of genes, proteins are complex structures that are made even more complicated by post-translational modifications such as splicing, methylation, phosphorylation and glycosylation. Because of these post-translational modifications, over tens of millions of proteins are generated from just over tens of thousands of genes. Proteins participate in the vast majority of biochemical processes in living organisms. Consequently, proteins are of great clinical interests as drugs and drug targets, as well as biomarkers for a variety of diseases. Using proteomics analysis, researchers are now attempting to correlate diseases and disease states to the presence or absence of a protein, or subset of proteins, and their post-translationally modified products. The ultimate goal of this proteomic research is to discover novel biomarkers that will uncover diagnostic and therapeutic targets.

With the advancement of ionization techniques such as electrospray ionization (ESI) and matrix assisted laser desorption-ionization (MALDI), mass spectrometry (MS) has emerged as a powerful tool for the analysis of proteins. In combination with one-dimensional (1-D) and two-dimensional (2-D) gel electrophoresis or liquid chromatography (LC) and capillary electrophoresis (CE), mass spectrometry has been successfully used to identify novel proteins through informatic tools such as peptide mass fingerprint, peptide sequence, or MS/MS ion search analysis, to name a few.

However, the tedious and complicated front-end sample preparation for MALDI and ESI mass spectrometry, by either 2-D gel electrophoresis, LC or CE has limited the MS application to mostly the research market only. An alternative technology, based on the surface enhanced laser desorption/ionization time-of-flight mass spectrometry (SELDI-TOF-MS), has been developed for potential clinical applications. Using SELDI chip technology, the sample preparation is accomplished by on-chip retentate chromatography, while detection is accomplished by TOF-MS. The SELDI chips, with varying chromatographic surfaces such as anion exchange, metal affinity, and reverse phase, can selectively bind to a class of proteins and present the proteins for SELDI-TOF analysis. The SELDI-TOF-MS technology has successfully identified ovarian cancer cases (E. F. Petricoin et al., Lancet 359: 572 (1992)).

In addition to qualitative identification of proteins, MS has been used to quantify the protein expression levels in tissues or plasma. Isotope-coded affinity tags (ICAT) (S. P. Gygi et al., Nature Biotech. 17: 994-999 (1999)) is a type of differential display that allows a direct quantitative comparison of relative protein expression between two or more samples by comparing the relative abundance of stable isotope versions of the same tag. However, since ICAT targets cysteine residues, proteins that lack cysteine (about 10% of all proteins) will be excluded by this approach. An alternative isotopic labeling method termed global internal standardization technology (GIST) (F. E. Regnier, et al., J Mass Spectrom. 37: 133 (2002)) has also been developed. The GIST strategy involves tryptic digestion of the control and experimental proteomes, followed by differential isotopic labeling.

Common approaches to GIST include ¹⁸O incorporation during digestion or N-acetoxy-¹³C₃-succinimide alkylation after digestion. After labeling, the two populations of digested proteins are mixed together and analyzed by 1-D or 2-D liquid chromatography-mass spectrometry. The intensity ratios of the co-eluting peptides from the light isotope-labeled peptide to the heavy isotope-labeled peptide are subsequently measured. Peptides pairs that have a ratio much greater than or less than unity are then flagged as a candidate biomarker. Like ICAT, GIST has been limited to the research market because of the tedious isotope labeling process.

Other efforts to develop a quantitative MALDI technique by addition of an internal standard (IS) with structures similar to the targeted protein have been developed. For the quantification of bovine insulin, a series of internal standards including horse heart cytochrome C, bovine insulin chain B etc. have been investigated (W. R. Wilkinson et al., J Anal. Chem. 357: 241 (1997)). Cytochrome C has also been disclosed in U.S. Published Patent Application 2002/0031773 as an internal standard in quantitative MALDI-TOF mass spectrometry of peptides and proteins. Similarly, to determine the concentration of sphingolipid in a test sample, an internal standard with a similar chemical structure, but having a different mass from the test sphingolipid has been employed. (See WO 03/048784A2). Most of the reported internal standards have found limited applications in the clinical market because of their species-specific nature.

Thus, there is a need in the art for a reliable, versatile internal standard for emerging protein analysis technology platforms.

### SUMMARY OF THE INVENTION

The present invention pertains to methods of quantifying the levels of at least one analyte in a sample or extract comprising adding a known quantity of at least one internal standard to the sample or extract using mass spectrometry. The present invention also relates to internal standards for mass spectrometry. Internal standards for mass spectrometry according to the invention can be used for many purposes, including assisting the alignment of mass spectra obtained from two different samples, each of which comprises the internal standard. The present invention also relates to compositions comprising at least one analyte, at least one internal standard and at least one matrix molecule, with the compositions being on a solid support.

In one embodiment, the internal standard is a dendrimer. Dendrimers are formed by an iterative sequence of reaction steps from polymer building blocks, giving the dendrimer macromolecules an advantageously consistent size, form and chemical reactivity. Dendrimers are resistant to proteases, and in one embodiment of the invention a dendrimer is used as an internal standard in a biological sample containing proteases. The dendrimer internal standard may be composed of building blocks including, but not limited to, poly(ethylene glycol) (PEG) or poly(amidoamine) (PAMAM).

According to one aspect, the present invention provides a method of quantifying a level of at least one analyte in a sample, the method comprising: (a) adding a known quantity of at least one dendrimer to the sample; (b) quantifying the levels of the analyte and dendrimer in the sample using mass spectrometry; (c) determining the difference between the known quantity of the dendrimer in (a) and the level of the dendrimer quantified in (b); and (d) correlating the difference of the dendrimer determined in (c) with the level of the analyte in (c).

In another embodiment of the invention, the sample comprises a dendrimer internal standard and may further comprise a labile internal standard that is sensitive to heat, pH, proteases, etc. The labile internal standard is partially degraded if the sample or extract is exposed to the conditions that cause the degradation of the labile internal standard. The degree of degradation of the labile internal standard reflects the degree of exposure of the sample or extract to conditions that cause the degradation of the labile internal standard.

According to another aspect of the invention, a composition comprises a dendrimer and at least one matrix molecule suitable for mass spectrometry. In one embodiment, the composition comprises a poly(ethylene glycol) (PEG) dendrimer or a poly(amidoamine) (PAMAM) dendrimer.

In yet another embodiment of the invention, an arrangement comprises a composition and a specimen collection container, where the composition comprises a dendrimer internal standard and the specimen collection container comprises an internal chamber that is partially evacuated or sterile.

The present invention further relates to a method for proteomic or other analysis where at least one internal standard is incorporated in a specimen collection device. The present invention also provides a method for proteomic analysis where at least one internal standard is used in combination with a protein chip capable of retaining, reacting with, or binding to at least one analyte.

### BRIEF DESCRIPTION OF THE DRAWINGS

FIG. 1 depicts mass spectra of different plasma concentrations containing constant levels of dendrimer as an internal standard.

FIG. 2 depicts mass spectra of plasma samples with 10% protease added and dendrimer samples with 10% protease added.

### DETAILED DESCRIPTION OF THE INVENTION

The invention relates to methods of quantifying the levels of at least one analyte in a sample or extract. The methods of the present invention comprise adding a known quantity of at least one internal standard to a sample or extract and determining the levels of the at least one analyte and the added internal standard(s) in the sample or extract, typically using mass spectrometry. After measuring the levels of the internal standard(s) and analyte(s), the difference between the levels of the internal standard(s) added to the sample or extract with the measured levels of the internal standard(s) is determined and correlated with the levels the analyte(s).

The invention also relates to methods to align mass spectra obtained from different samples. Spectra from two or more samples, each of which comprise the internal standard, are aligned by reference to a known physical characteristic of the internal standard, such as the time of flight or the m/Z value of the internal standard.

As used herein, the term “quantify” can be used to mean determining the absolute or relative levels of a particular analyte. The quantity can be expressed as a difference in two values, a percentage, ratio or absolute change between two or more sets of experimental variables, or absolute or relative levels of an analyte. The quantity may or may not be expressed in any unit of measure.

The analyte to be quantified can be known or unknown. That is, the analyte to be quantified, for example, a protein, need not have its specific biochemical properties known prior to the methods of the current invention. In fact, the methods described herein can be used to delineate the biochemical properties of an unknown analyte. Alternatively, the methods described herein can be used to quantify a previously characterized, partially or complete, analyte. Because the methods described herein can be used to quantify more than one analyte simultaneously, the analytes quantified need not be pure. Examples of analytes that can be quantified using the methods described herein include, but are not limited to, proteins, polypeptides, oligopeptides, amino acids, monosaccharides, disaccharides, polysaccharides, nucleotides, oligonucleotides, polynucleotides, proteoglycans, glycoproteins, lipids, lipoproteins, natural polymers and soluble synthetic polymers. There may be one, two, three, four, five or more analytes in a single sample or extract that are quantified.

The methods described herein allow absolute or relative quantification across multiple samples or extracts. For example, the ratio of the measured analyte to the measured internal standard, or its reciprocal, may be calculated and compared within or among the same or different samples. The multiple samples or extracts used can simply be more than one of the same sample or extract type, e.g., more than one identical cell culture or body tissue, or they can be different samples or extracts, e.g., two different cell cultures or two different cell body tissues or fluids. Furthermore, the different samples or extracts can be derived from more than one individual animal, plant or microorganism species. Thus, the difference(s) in levels or ratios of the samples or extracts can, for example, derive from experimental variables, different individuals or different tissues or fluids within the same individual.

As used herein, the term “sample” is used to mean at least a portion of a solid, liquid or gas to be analyzed. The terms “specimen” and “sample” are used interchangeably herein. The sample can be biological, chemical or environmental in nature. Examples of environmental samples include, but are not limited to, samples taken from oil, soil and water. A chemical sample can be organic or inorganic. Examples of chemical samples also include chemicals for human use or consumption, such as food and cosmetics. In one embodiment, the sample is a biological sample. Examples of biological samples include, but are not limited to, a cell culture, an animal tissue and a body fluid. The cells in the cell culture can be animal cells, plant cells, bacterial cells and fungal cells. If the sample is composed, in part or in whole, of animal cells, the animal from which the cells ultimately derive can be from any vertebrate or nonvertebrate animal, such as a mammal including, but not limited to, a rodent, bovine, equine, canine, feline, porcine, human and non-human primates.

When a sample is used, the invention may also encompass a processing step to produce an extract from the sample. The extract can be produced by any means necessary, provided that the extract contains the analyte to be quantified. The extract can be in solid, liquid or gas form and need not be pure. Furthermore, the extract-producing processing step may concentrate or even dilute the analyte to be quantified, or it may chemically modify the analyte to render it, for example, more or less hydrophobic, more or less hydrophilic or more or less ionic. The internal standard used in the current invention can be placed in the sample before, during or after the extract-producing processing described herein.

As used herein, the term “internal standard” refers to what is added to and subsequently detected and quantified in the sample or extract. The addition of the internal standard can be before, during or after sample or extract collection or processing. Furthermore, as discussed herein, “addition of internal standard” also encompasses collection devices that are manufactured to include internal standard(s) prior to their use, either contained therein or otherwise associated therein (e.g., as a kit). The internal standard, as contemplated by the present invention, is a compound that is added to the sample or extract, and this identical compound is then quantified using the methods described herein.

In one embodiment of the current invention, the internal standard used is a dendrimer. As understood in the art, a “dendrimer” is a large molecule of a discrete size with a regular and highly branched three-dimensional structure that is built by an iterative sequence of reaction steps from primary building blocks such as, for example, polymers.

Polymers, which can serve as building blocks for the dendrimers, are generally classified in a structural sense as either linear or branched. In the case of linear polymers, the repeating units (often called “mers”) are divalent and are connected one to another in a linear sequence. In the case of branched polymers, at least some of the mers possess a valency greater than two such that the mers are connected in a nonlinear sequence. The term “branching” typically means that the individual molecular units of the branches are discrete from the polymer backbone, yet have the same chemical constitution as the polymer backbone. Thus, regularly repeating side groups that are inherent in the monomer structure and/or are of different chemical constitution than the polymer backbone are not considered as branches, e.g., dependent methyl groups of linear polypropylene. To produce a branched polymer, an initiator, a monomer, a “functional core” or any combination thereof that possesses at least three moieties that function in the polymerization reaction, may be required. Such monomers or initiators are often called polyfunctional. The simplest branched polymers are the chain branched polymers wherein a linear backbone bears one or more essentially linear pendant groups. This simple form of branching, often called “comb branching,” may be regular, wherein the branches are uniformly and regularly distributed on the polymer backbone, or irregular, wherein the branches are distributed in nonuniform or random fashion on the polymer backbone. See T. A. Orofino, Polymer 2: 295-314 (1961); T. Altores et al. in J. Polymer Sci., Part A, Vol. 3, pp. 4131-51 (1965) and Sorenson et al. in “Preparative Methods of Polymer Chemistry”, 2nd Ed., Interscience Publishers, 213-214 (1968), which are hereby incorporated by reference.

Another type of branching is exemplified by cross-linked or network branched polymers wherein the polymer chains are connected via tetravalent compounds, e.g., polystyrene molecules bridged or cross-linked with divinylbenzene. In this type of branching, many of the individual branches are not linear in that each branch may itself contain groups pendant from a linear chain. More importantly in network branching, each polymer macromolecule (backbone) is cross-linked at two or more sites to two other polymer macromolecules. Also, the chemical constitution of the cross-linkages may vary from that of the polymer macromolecules. In this so-called cross-linked or network branched polymer, the various branches or cross-linkages may be structurally similar (called “regular cross-linked”) or they may be structurally dissimilar (called irregularly cross-linked). An example of regular cross-linked polymers is a ladder-type poly(phenylsilsesquinone) as described by Sorenson et al., supra, at 390. The foregoing and other types of branched polymers are described by H. G. Elias in Macromolecules, Vol. I, Plenum Press, New York (1977), which is hereby incorporated by reference.

Additionally, there are polymers having so-called star-structured branching wherein the individual branches radiate out from a nucleus and there are at least three branches per nucleus. The outermost tier or generation of the star dendrimers terminates in functional groups that may be chemically reactive with a variety of other molecules. Thus, star dendrimers are unitary molecular assemblages that possess three distinguishing architectural features, namely (a) an initiator core, (b) interior layers (generations) composed of repeating units radially attached to the initiator core, and (c) an exterior surface of activated functional groups attached to the outermost, or terminal, ends of each branch. Such star-branched polymers are illustrated by the polyquaternary compositions described in U.S. Pat. Nos. 4,036,808 and 4,102,827, which are hereby incorporated by reference. Star-branched polymers prepared from olefins and unsaturated acids are described in U.S. Pat. No. 4,141,847, which is hereby incorporated by reference. Star-branched polymers are often less sensitive to degradation. Additionally, the star-branched polymers have relatively low intrinsic viscosities, even at high molecular weight.

The size, shape and reactivity of a dendrimer can be controlled by the choice of the initiator core, the number of generations employed in creating the dendrimer, and the choice of the repeating units employed at each generation. Dendrimers of discrete sizes are readily obtained as the number of generations employed increases. Examples of dendrimers that can be used in the methods described herein include, but are not limited to, those described in U.S. Pat. No. 6,455,071, which is incorporated herein by reference.

The spherical dendrimers by their very nature may interact with or bind to substances on a protein chip surface in a much more predictable and reproducible fashion. Examples of spherical dendrimers of configurations suitable for use in the present invention are disclosed in U.S. Pat. No. 4,507,466 and U.S. Pat. No. 4,568,737, both of which are incorporated herein by reference. Alternatively, dendrimers of non-spherical configuration, such as those disclosed in U.S. Pat. No. 4,694,064, incorporated herein by reference, may be adapted for use in the present invention.

Dendrimers that can be used in accordance with the present invention include, but are not limited to, a dendritic macromolecule of the polyester type as disclosed in U.S. Pat. No. 5,418,301, a carbosilane-based hybrid star polymer as disclosed in U.S. Pat. No. 5,276,110, an epoxide-amine dendrimer as disclosed in U.S. Pat. No. 5,760,142, a silicon-containing multi-arm star polymer as disclosed in U.S. Pat. No. 6,350,384, a saccharide-containing dendrimer disclosed in U.S. Pat. No. 6,417,339, a dendritic and highly branched polyurethane disclosed in U.S. Pat. No. 6,376,637, a quaternary ammonium functionalized dendrimer disclosed in U.S. Pat. No. 6,440,405, a calixarene conjugate disclosed in U.S. Pat. No. 5,622,687, a polypeptide dendrimer disclosed in U.S. Pat. No. 4,558,120, and any combination thereof. The present invention also encompasses any chemical modifications and variations of the cited dendrimers.

In another embodiment of the invention, a “labile internal standard” that is not a dendrimer is used in the same sample or extract as the dendrimer. The labile internal standard is more sensitive to heat, pH, proteases, etc. than the dendrimer internal standard, so that the labile internal standard is partially degraded if the sample or extract is exposed to those conditions that cause the degradation of the labile internal standard. The degree of degradation of the labile internal standard reflects the degree of exposure of the sample or extract to conditions that cause the degradation of the labile internal standard. The labile internal standard may be used to monitor processing, handling, collection, storage, transport and/or extraction procedures performed on the sample or extract. For example, the labile internal standard may partially degrade in the presence of proteases. Degradation of the labile internal standard in a sample or extract thus would indicate that the sample or extract had been exposed to proteases. The labile internal standard likewise may degrade partially in response to conditions such as, but not limited to, high or low pH, extreme salt concentrations, heat, and any other condition occurring during the processing or concentration methods to which the sample or extract is subjected. Labile internal standards according to the present invention include, but are not limited to, [Arg8]-vasopressin, [Glu1]-fibrinogen, ACTH [1-24], albumin, angiotensin, beta-endorphin [61-91], beta-galactosidase, beta-lactoglobulin A, carbonic anhydrase, conalbumin, cytochrome C, dynorphin A [209-225], GAPDH, Hirudin BKHV, IgG, insulin, insulin, insulin B-chain, myoglobin, peroxidase, somatostatin, superoxide dismutase, ubiquitin and/or any combination thereof.

As used herein, “proteomic analysis” is an assay that characterizes, confirms the identity of, identifies, or discovers the existence of unknown or known proteins in a sample or extract. Furthermore, “mass spectrometry” is intended to mean any technology platform that detects separate individual components of a sample based on their relative or absolute masses. The detection of the sample components, based on their mass, can then be used to identify, characterize or confirm the identity of at least one of the components of the sample. The detection of the sample components can be based on, for example, the electrical charge of the individual components and the time that the individual components require in traveling between at least two points. Generally speaking, mass spectrometry is coupled with an additional technology platform that will initially separate the sample components in such a way that they may be detected. This additional technology platform often comprises an energy source that urges the sample components from a more grounded state. Examples of these additional technology platforms that energize the sample include, but are not limited to, electrospray ionization (ESI) and laser desorption/ionization-type spectrometry.

As used herein, “laser desorption/ionization-type spectrophotometry” is used to mean any type of analytical tool that uses an energy source, for example, a laser, to cause or force molecules to dissociate (desorb) from a support. For example, lasers can be used to cause desorption of molecules from a solid support. Additional examples of using a laser to cause molecules to dissociate from a support include vaporization and sublimation. The support from which the molecules are dissociating can be solid or liquid supports. Accordingly, examples of laser desorption/ionization-type spectrophotometry include, but are not limited to, matrix-assisted laser desorption/ionization (MALDI) and surface enhanced laser desorption/ionization (SELDI). The laser desorption/ionization-type spectrophotometry can then be combined with any type of assay to analyze the molecules being dissociated from the support. For example, as used herein, laser desorption/ionization-type spectrophotometry can be further combined with such types of analyses as including, but not limited to, high pressure liquid chromatography (HPLC), gas chromatography (GC), quadrupole spectroscopy (O) and time-of-flight spectroscopy (TOF). Examples of laser desorption/ionization-type spectrophotometry combined with other types of analyses include, but are not limited to, MALDI-TOF, SELDI-TOF, MALDI-TOF-MS, SELDI-TOF-MS, SELDI-Q-TOF and MALDI-MS/MS.

According to the present invention, using laser desorption/ionization-type spectrophotometry to determine the levels of analyte(s) and internal standard(s) means that laser desorption/ionization-type spectrophotometry may be used in at least one step of the determination. For example, it is possible that mass spectroscopy may be the tool that actually generates the quantitative data to determine levels of the analyte(s) and internal standard(s). However, if the MS analysis is coupled, in any way, to laser desorption/ionization-type spectrophotometry, for example, MALDI, this type of MALDI-MS analysis is within the scope contemplated by the term “laser desorption/ionization-type spectrophotometry.”

Alternatively, electrospray ionization passes an electrical charge to the sample, be it solid or liquid, to a very high voltage. The sample becomes increasingly unstable as the voltage of the sample increases with time and electrical current. The increasing instability ultimately results in the sample breaking apart into a very fine mist, where the droplets are less that about 10 μm.

Additional technology platforms or analyses that can be used in the present invention include such techniques as high performance liquid chromatography (HPLC), gel electrophoresis, gas chromatography (GS), infrared spectrophotometry (IR), ultraviolet spectrophotometry (UV), nuclear magnetic resonance spectroscopy (NMR), SDS-PAGE, isoelectric focusing, Western blot and capillary electrophoresis.

As used herein, “difference” is any difference in levels of internal standard(s) and analyte(s) that is a discernable or statistical difference, whether it is a relative or absolute difference. The differences in levels need not be statistically significant. Furthermore, the difference in measured internal standards can be expressed numerically or qualitatively or, for example, as ratios of internal standard to analyte or of one internal standard to another internal standard. The scope of the invention should not be limited by the statistical method of producing the discernable differences or ratios involving the internal standard(s).

The difference in the levels of internal standard(s) is then correlated to the levels of analyte(s) detected. The correlation process can be any process used to create a relationship between the levels of the internal standard(s) and the analyte(s). For example, the correlation may be arithmetic, geometric or logarithmic. The correlation may be a simple ratio between two values, or the difference in the internal standard may be used in a more complex algorithm to determine the levels of the analyte. The differences or ratios are useful in not only determining absolute or relative levels of the analyte(s), but they may also be useful in monitoring various quality control aspects of, for example, sample collection, processing and/or testing.

The present invention also relates to internal standards used in laser desorption/ionization-type spectrophotometry. As contemplated by the present invention, the scope of “internal standard” has previously been described herein. In one embodiment, the internal standard is a polymer or dendrimer, such as, but not limited to, various dendrimers of poly(amidoamine) (PAMAM), poly(ethylene glycol) or combinations thereof.

In one embodiment, the internal standard assists in the alignment of mass spectra obtained from different samples. Small instrumental variations may cause ions to have slightly different times-of-flight, for example, from one experiment to the next. The addition of the internal standard of the invention to samples that are analyzed at different times advantageously allow an investigator to correct for these instrumental variations by referring to the time-of-flight of the internal standard as an internal control. This embodiment is especially advantageous when the investigator is comparing samples comprising different complements of ions.

As used herein, “PAMAM dendrimer” is used to mean any dendrimer where poly(amidoamine) is the core molecule, regardless of the generation number, the number of arms and the constitution of the arms. Furthermore, the PAMAM dendrimer can be any shape or structure, such as, but not limited to, stars or star-branched polymers. Likewise, “PEG dendrimer” dendrimer where is used to mean any dendrimer where poly(amidoamine) is the core molecule, regardless of the generation number, the number of arms and the constitution of the arms. The PEG dendrimers used in the current invention can also be any shape or structure. The preparation and characterization of PAMA/PEG dendrimers is described in Hedden and Bauer, Macromolecules, 36:1829-35 (2003), which is hereby incorporated by reference.

The following tables list some commercially available (Aldrich, Milwaukee, Wis.) PAMAM dendrimers and their molecular weights.

Table 1 shows PAMAM dendrimers.

| Generation | Molecular Weight (Dalton) |
|---|---|
| −0.5 | 436.28 |
| 0 | 516.69 |
| 0.5 | 1,269.06 |
| 1 | 1,429.88 |
| 1.5 | 2,935 |
| 2 | 3,256 |
| 2.5 | 6,267 |
| 3 | 6,909 |
| 3.5 | 12,931 |
| 4 | 14,215 |
| 4.5 | 26,258 |
| 5 | 28,825 |
| 5.5 | 52,901 |
| 6 | 58,047 |
| 6.5 | 106,198 |
| 7 | 116,491 |
| 7.5 | 212,793 |
| 8 | 233,378 |
| 9 | 467,151 |
| 10 | 934,698 |

Table 2 shows PAMAM-OH dendrimers.

| Generation | Molecular Weight (Dalton) |
|---|---|
| 2 | 3,272 |
| 3 | 6,941 |
| 4 | 14,279 |
| 5 | 28,951 |
| 6 | 58,299 |
| 7 | 116,995 |

In one embodiment, the terminal groups of PAMAMs are modified during synthesis, rendering the resulting dendrimers capable of binding to protein chips with surface properties such as, for example, hydrophobic, hydrophilic, anionic, cationic, or metal-binding. For example, both hexylamine capped generation 1.5 of PAMAM with a formulation of N(CH₂CH₂CONCH₂CH₂N(CH₂CH₂CONHCH₂CH₂N(CH₂CH₂CONHCH₂CH₂CH₂CH₂CH₂CH₃)₂)₂)₂)CH₂CH₂N(CH₂CH₂CONHCH₂CH₂N(CH₂CH₂CONHCH₂CH₂N(CH₂CH₂CONHCH₂CH₂CH₂CH₂CH₂CH₃)₂)₂)₂ and methylamine capped generation 1.5 of PAMAM with a formulation of N(CH₂CH₂CONHCH₂CH₂N(CH₂CH₂CONHCH₂CH₂N(CH₂CH₂CO—NHCH₃)₂)₂)₂)CH₂CH₂N(CH₂CH₂CONHCH₂CH₂N(CH₂CH₂CONHCH₂CH₂N(CH₂CH₂CONHCH₃)₂)₂)₂ can consistently bind to both a weak cationic exchange (WCX-2 from Ciphergen) and a hydrophobic chip (H50 from Ciphergen) protein chip. Examples for the use of the methylamine capped poly(amidoamine) dendrimer as an internal standard will be presented in the invention.

The present invention also relates to composition of matter, found on a solid support, comprised of at least one internal standard, as described hereinabove, and at least one matrix molecule.

As used herein, a “matrix molecule” refers to a natural or synthetic molecule or to a compound or polymer that can crystallize on a solid surface. The crystallization can take place on the solid support, or the matrix molecule can be applied, as a crystal to the solid support. In one embodiment, the matrix molecule is an organic molecule. Examples of matrix molecules include, but are not limited to, sinapinic acid (SPA), alpha-cyano-4-hydroxycinnamicacid (CHCA), 2,5-dihydroxybenzoicacid (DHB), 3-hydroxypicolinic acid, 2′,4′,6′-trihydroxyacetophenone (THAP), 2-(4-hydroxyphenylazo)benzoic acid, succinic acid, anthranic acid, nicotinic acid, salicylamide, isovanillin, 3-aminoquinoline, 1-sioquinolinol and Dithranol.

Examples of solid supports include, but are not limited to, any chip (for example, silicon-based, metal, ceramic, glass, or gold chip), glass slide, membrane, bead, solid particle (for example, agarose, sepharose, or magnetic bead), column (or column material), test tube, microtiter dish or the like. The solid support can be made from a variety of materials including, but not limited to, glass, nylon, polymethacrylate, polystyrene, polyvinylchloride, latex, chemically modified plastic and rubber.

The present invention also provides methods for proteomic analysis where at least one internal standard is directly incorporated into a specimen collection device prior to the specimen collection. The incorporation of the internal standard into a specimen collection device is accomplished before, during, or after the specimen collection device is manufactured. The specimen collection device can be used to collect any specimen of biological, chemical or environmental nature. In one embodiment, the specimen collection device contains an internal standard that is a dendrimer. In another embodiment, the specimen collection device further contains an additional internal standard that is a labile internal standard.

The internal standard may be located on any surface of the collection device. The internal standard may also be located on stoppers and seals for closing such devices or on mechanical, or other, inserts placed within such devices. The internal standard can be located anywhere along at least one interior wall of the collection device or anywhere within the reservoir portion. It may be also desirable to protect the internal standard from light, in the event the internal standard is light sensitive. For such internal standard, use of an opaque tube, e.g., an amber-colored tube, would be encompassed by the invention herein. Alternatively, placing the internal standard into a capsule that protects it from light exposure, e.g., in powdered form, and then placing the capsule into the tube would also fall within the scope of the invention.

The internal standard may be applied to the collection device by any number of methods. For example, the internal standard may be spray dried, loosely dispensed or lyophilized over the surface of the interior wall of the collection device. Alternatively, the internal standard, such as when in gel or liquid form, for example, may be positioned in the reservoir portion of the collection device. Additional methods for providing the collection device with the internal standard are also possible. One method of depositing the desired amount of internal standard into a collection device is to reconstitute or dissolve a solid form of the internal standard into a solvent and then dispense the solution into the collection device. The liquid may be spray dried, disposed into the bottom of the container or subsequently lyophilized.

The quantity and location of the internal standard are determined by several variables, including the mode of application, the specific internal standard used, the internal volume and internal pressure of the collection device, and the volume of the biological sample drawn into the container.

In one embodiment, the specimen collection device comprising the internal standard further comprises at least one preservative, additive and/or stabilizer. Examples of a preservative, additive and/or stabilizer include an anticoagulant, such as EDTA, heparin, citrate, a procoagulant, an antimicrobial agent, an antioxidant and a protease inhibitor. “EDTA” for the purposes of the present invention includes the free acid, metal chelates and salts of EDTA, such as the disodium, dipotassium, and tripotassium salts. The internal standard is selected to be physical and chemically compatible with the preservative, additive and/or stabilizer. Selecting a preservative, additive and/or stabilizer that is compatible with the internal standard is a matter of routine optimization and experimentation in the art. The internal standard in the specimen collection device may be either mixed or combined with the preservative, additive and/or stabilizer or may be partitioned from the same. In one embodiment, the internal standard is stable enough to be incorporated in the manufacture, transportation, and eventual usage of the specimen collection device. An example of such a specimen collection device, comprising protease inhibitors, is discussed in U.S. patent application Ser. No. 10/436,236, hereby incorporated by reference. Other uses of preservatives, additives and/or stabilizers in accord with the present invention would be apparent to those skilled in the art.

In one embodiment, the stabilizer is a protease inhibitor. Suitable examples include, but are not limited to, inhibitors of serine proteases, cysteine proteases, aspartic proteases, metalloproteases, thiol proteases, exopeptidases, and the like. Non-limiting examples of serine protease inhibitors include antipain, aprotinin, chymostatin, elastatinal, phenylmethylsulfonyl fluoride (PMSF), APMSF, TLCK, TPCK, leupeptin and soybean trypsin inhibitor. Inhibitors of cysteine proteases include, for example, IAA (indoleacetic acid) and E-64. Suitable examples of aspartic protease inhibitors include pepstatin and VdLPFFVdL. Non-limiting examples of inhibitors of metalloproteases include EDTA, as well as 1,10-phenanthroline and phosphoramodon. Inhibitors of exopeptidases include, for example, amastatin, bestatin, diprotin A and diprotin B. Additional suitable examples of protease inhibitors include alpha-2-macroglobulin, soybean or lima bean trypsin inhibitor, pancreatic protease inhibitor, egg white ovostatin and egg white cystatin. Combinations of protease inhibitors, referred to as a “protease inhibitor cocktail,” may also be used as the stabilizing agent. Such “cocktails” are generally advantageous in that they provide stabilization for a range of proteases; therefore, a stabilizing agent containing more than two protease inhibitors is generally desirable.

The sample collection system of the present invention can encompass any collection device including, but not limited to, tubes such as test tubes and centrifuge tubes; closed system blood collection devices, such as collection bags; syringes, especially pre-filled syringes; microtiter and other multi-well plates; arrays; laboratory vessels such as flasks, spinner flasks, roller bottles, vials; tissue and other biological sample collection containers; and any other container suitable for holding a biological sample, as well as containers and elements involved in transferring samples. As used herein, “collection device” and “collection container” are used interchangeably. In one embodiment of the current invention, the sample collection system comprises a separating member, e.g., a mechanical separating element or a gel, for separating blood components. In such aspect, the interior of the tube and/or the exterior of the separating member may be treated with the internal standard and/or other compound such as a stabilizer, preservative or additive. The separator may be used to separate, e.g., plasma, serum, or particular cell types, upon centrifugation. Tubes containing other separating elements are also possible.

A useful manufacturing process for devices according to the present invention involves obtaining a collection container; adding at least one internal standard to the container; lyophilizing the at least one internal standard; evacuating the container; and sterilizing the container. The at least one internal standard may be dispensed into the container in solution form. Additional reagents, such as stabilizers, can also be added to the container at this time. After adding the internal standard to the collection container, a separating member may be added to the container, if desired. An example of a suitable lyophilization/evacuation process is as follows: the container is frozen at a temperature of about −40° C. at a pressure of about 760 mm for about 6 to 8 hours; the container is dried as the temperature is ramped from −40° C. to about 25° C. at a pressure of about 0.05 mm for about 8 to 10 hours; and the container is then evacuated at a temperature of about 25° C. and a pressure of about 120 mm for about 0.1 hours. The sterilization technique can be accomplished with, for example, cobalt-60 radiation.

A collection device comprising at least one internal standard is useful in any analytical technique, e.g., those listed herein, for quantifying the amount of an analyte present in the sample. The presence of the internal standard in the collection device prior to sample collection reduces the number of handling or processing steps required to analyze the sample, thereby reducing the number of times during which the sample may be lost and/or contaminated. For example, a collection device comprising at least one internal standard could be used to collect a biological sample (e.g., blood) that is to be analyzed via HPLC, GC, or a MS-related analysis.

Plastic or glass is often used to manufacture the collection device; however, the scope of the collection devices herein is not limited by the materials used in their manufacture. Examples of materials used in the manufacture of the collection devices include, but are not limited to, polypropylene, polyethylene, polyethyleneterephthalate, polystyrene, polycarbonate, cellulosics, polytetrafluoroethylene and other fluorinated polymers, polyolefins, polyamides, polyesters, silicones, polyurethanes, epoxies, acrylics, polyacrylates, polysulfones, polymethacrylates, PEEK, polyimide and fluoropolymers such as PTFE Teflon®, FEP Teflon®), Tefzel®, poly(vinylidene fluoride), PVDF and perfluoroalkoxy resins. The collection devices may also be composed of glass, including but limited to, silica glass. For example, PYREX® (available from Corning Glass, Corning, N.Y.) may be used in the manufacture of the collection devices. Furthermore, ceramic and cellulosic products such as paper and reinforced paper containers can be used in the manufacture of the specimen collection devices.

The present invention also provides a method for proteomic analysis where at least one internal standard is used in combination with a protein chip cable of retaining, reacting, or binding to at least one analyte, such as a protein. Examples of such a protein chip can be found in U.S. Pat. No. 6,579,719 and U.S. Published Patent Application Nos. 2002/0177242 and 2002/0155509, all of which are hereby incorporated by reference. In one embodiment, the chip has a retentate chromatographic surface that can bind, retain or react with at least one type of analyte. Examples of retentate surfaces that comprise the protein chip include, but are not limited to, an anion, a cation, a hydrophobic interaction adsorbent, a metal ion, a reducing agent, a polypeptide, a nucleic acid, a carbohydrate, a lectin, a dye, a hydrocarbon, a polymer or a combination thereof. In one embodiment, the internal standard will consistently interact with the protein chip when mixed with the targeted analytes. The protein chip, comprising the retentate surface and the internal standard(s), can be read on a mass spectrometer such as, but not limited to, MALDI-TOF, SELDI-TOF, MALDI-TOF-MS, SELDI-TOF-MS, SELDI-Q-TOF, MALDI-MS/MS, or any modification or combination thereof. By maintaining a constant amount or concentration of internal standard between and among protein chip(s), the analyte(s) can be quantitatively compared among chips as well as among mass spectrometers.

### EXAMPLES

### Example 1

### Preparation of Plasma with Internal Standard

Poly(amidoamine) (“PAMAM”), generation 2, with methylamine surface (at 13.8%, w/w) can be purchased from Dendritech, Midland, Mich. To prepare a working solution of dendrimer to be added to a blood sample, 100 μl of 1.4% dendrimer was added to 900 μl of 50% acetonitrile.

Plasma (500 μL) was prepared from blood collected in a BD Vacutainer® PPT™ tube (available from Becton, Dickinson and Company, Franklin Lakes, N.J.) per conventional protocol and was kept at −80° C. After thawing, the plasma was divided and diluted with the WCX-2 binding buffer (20 mM ammonium acetate and 0.1% TFA, pH 6) according to Table 3. After dilution, 10 μL of the internal standard dendrimer (1.38%) in acetonitrile/de-ionized water (1:1) was added to the plasma. The final concentration of the internal standard was kept constant for all the plasma dilution samples.

TABLE 3
Plasma preparation conditions

| Sample | Plasma | Dendrimer | Plasma | WCX2 |
|---|---|---|---|---|
| # | Dilution | (μL) | (μL) | Buffer (μL) |
| 1 | 1:10 | 10 | 10 | 80 |
| 2 | 1:50 | 10 | 2 | 88 |
| 3 | 1:100 | 10 | 1 | 89 |
| 4 | 1:500 | 10 | 2* | 88 |
| 5 | Control | 10 | — | 90 |

*Plasma diluted 1:10 in water, prior to use.

The WCX-2 chip (Ciphergen, Biosystems, Inc., Fremont, Calif.) was prepared in a Ciphergen bioprocessor according to the manufacturer protocol on a Biomek 2000 robot (Beckman Coulter). One WCX-2 chip has eight binding spots. The spots on the chip were successively washed twice with 50 μL of 50% acetonitrile for 5 minutes and then washed with 50 μL of 10 mM of HCl for 10 minutes and with 50 μL of de-ionized water for 5 minutes. After washing, the chip was conditioned twice with 50 μL of WCX-2 buffer for 5 minutes before the introduction of plasma samples.

To each spot on the conditioned WCX-2 chip, 100 μL of the prepared plasma samples were added manually. After incubation at room temperature for 30 minutes with shaking, the spots were then washed with 100 μL of the WCX-2 binding buffer twice, followed by a wash with 100 μL of de-ionized water twice. The chip was then dried and spotted twice with 0.75 μL of saturated solution of α-cyano hydroxy cinnamic acid (99%) (CHCA) or sinapic acid (SPA), in a 50% acetonitrile, 0.5% TFA aqueous solution.

The chips with bound plasma proteins were then read by SELDI-TOF-MS using the experimental conditions shown in Table 4.

TABLE 4
SELDI-TOF-MS reading conditions

| Experimental Settings | Matrix: SPA | Matrix: CHCA | |
|---|---|---|---|
| Detector Voltage | 2850 V | 2850 V | 2850 V |
| Deflector Mass | 1000 Da | 1000 Da | 1000 Da |
| Digitizer Rate | 500 MHz | 500 MHz | 500 MHz |
| High Mass | 75,000 Da | 75,000 Da | 75,000 Da |
| Focus Mass | 6000 Da | 30,000 Da | 30,000 Da |
| Intensity (low/high) | 200/205 | 160/165 | 145/150 |
| Sensitivity (low/high) | 6/6 | 6/6 | 6/6 |
| Fired/kept spots | 91/65 | 91/65 | 91/65 |

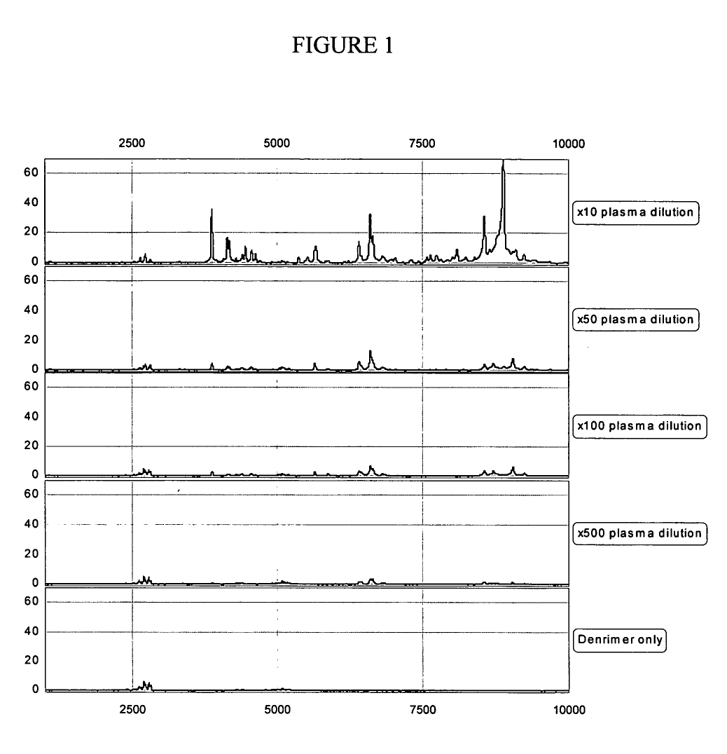

As it is shown in FIG. 1, the ion current intensities of the plasma proteins in the mass spectra were directly proportional to the concentration of the plasma samples. The triplet peaks (m/Z) centered around 2715 Da is the dendrimer. The peak is in triplet because of the incomplete methylation of the Starburst PAMAM dendrimer G (2.0). Note that the triplet dendrimer peak appeared in all five spectra, clearly indicating that the dendrimer is consistently bound on the WCX-2 chip and subsequently detected during SELDI-TOF-MS, as intended. The consistent peak heights for the dendrimer in all five spectra demonstrate that dendrimer can be used as internal standard. A dendrimer that produces a single peak may also be used as an internal standard. Furthermore, the internal standard can have multiple peaks, in any number, as indicted here.

### Example 2

### Effect of Protease on Dendrimer as an Internal Standard

Plasma (500 μL) was prepared from blood collected in a BD Vacutainer® PPT™ tube (Becton, Dickinson and Company, Franklin Lakes, N.J.) per conventional protocol and was kept at −80° C. After thawing, the plasma was divided and diluted with the WCX-2 binding buffer (20 mM ammonium acetate and 0.1% TFA, pH 6) according to Table 5. Then, 10 μL of internal standard dendrimer in acetonitrile/de-ionized water (1:1) was used. The protease solution (10%) in 0.1% trifluoroacetic acid was added to both the plasma and dendrimer solutions. The four samples prepared according to Table 3 were incubated at room temperature for 3 hours, followed by the WCX2 protocol using a Biomek Coulter 2000 robot.

TABLE 5
Plasma/Dendrimer Sample Preparation with and without Protease

| Sample | Dendrimer | Plasma | 10% Protease | WCX2 |
|---|---|---|---|---|
| # | (μL) | (μL) | (μL) | Buffer (μL) |
| 1 | 10 | — | — | 90 |
| 2 | — | 10 | — | 90 |
| 3 | 10 | — | 5 | 85 |
| 4 | — | 10 | 5 | 85 |

To each spot on the conditioned WCX-2 chip, 100 μL of the plasma or dendrimer samples were added manually. After incubation at room temperature for 30 minutes with shaking the spots were then washed with 100 μL of the WCX-2 binding buffer twice, followed by 100 μL of de-ionized water twice. The chips were then dried and spotted twice with 0.75 μL of the saturated solution of CHCA or SPA, in a 50% acetonitrile, 0.5% TFA aqueous solution.

The processed chips with plasma protein on the surface were then read by SELDI-TOF-MS, using the experimental conditions shown in Table 4, above.

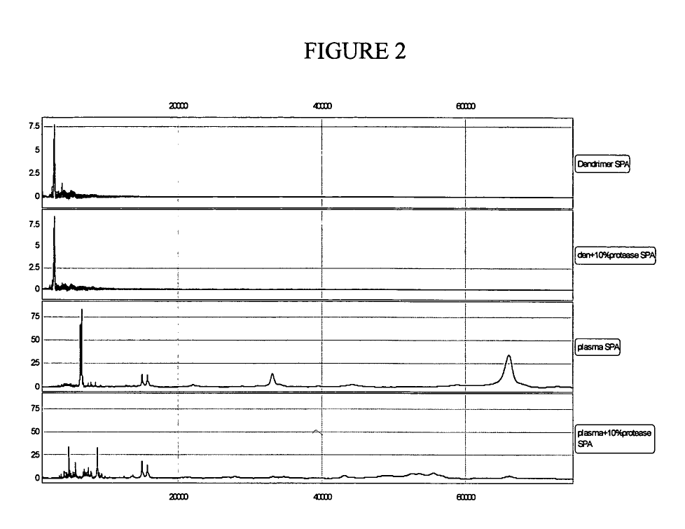

The SELDI spectra of dendrimer, dendrimer with 10% protease, plasma and plasma with 10% protease are shown in FIG. 2. By comparison, the first two spectra demonstrate that dendrimer remained unchanged after three-hour incubation with protease. On the other hand, significant degradation occurred when the plasma was incubated with the same concentration of protease for the same period of time. Thus, the addition of protease had no effect over the quantitative estimation of dendrimer as an internal standard, as the dendrimer remained stable, while the plasma proteins were degraded by the protease.

The foregoing detailed description of the preferred embodiments of the invention exemplifies principles of the invention and does not limit the invention to the disclosed specific embodiments. A skilled artisan may make numerous variations of these embodiments without departing from the spirit of the invention.

1. A method of quantifying a level of at least one analyte in a sample, the method comprising:
a) adding a known quantity of at least one dendrimer to the sample;
b) quantifying the level of the at least one analyte and the at least one dendrimer in the sample using mass spectrometry;
c) determining the difference between the known quantity of the at least one dendrimer in a) and the level of the at least one dendrimer quantified in b); and
d) correlating the difference of the at least one dendrimer in c) with the level of the at least one analyte in c).

2. The method of claim 1, further comprising preparing an extract of the at least one analyte from the sample prior to b).

3. The method of claim 1, wherein the sample is a biological sample.

4. The method of claim 3, wherein the biological sample is a cell culture, an animal tissue, or a body fluid.

5. The method of claim 4, wherein the biological sample is a cell culture comprising animal cells, plant cells, bacterial cells, or fungal cells.

6. The method of claim 1, wherein the at least one analyte comprises a protein, polypeptide, oligopeptide, amino acid, monosaccharide, disaccharide, polysaccharide, nucleotide, oligonucleotide, polynucleotide, proteoglycan, glycoprotein, lipid, lipoprotein, natural polymer or soluble synthetic polymer.

7. The method of claim 1, wherein the dendrimer is a poly(ethylene glycol) (PEG) dendrimer or a poly(amidoamine) (PAMAM) dendrimer.

8. The method of claim 7, wherein the mass spectrometry of step b) comprises laser desorption/ionization-type spectrophotometry.

9. The method of claim 8, wherein the laser desorption/ionization-type spectrophotometry comprises matrix-assisted laser desorption/ionization (MALDI), surface enhanced laser desorption/ionization (SELDI), MALDI-time-of-flight (MALDI-TOF), SELDI-TOF, MALDI-TOF-mass spectrometry (MS), SELDI-TOF-MS, SELDI-Q-TOF, SELDI-MS/MS, or any combination thereof.

10. The method of claim 7, wherein the mass spectrometry comprises electrospray ionization.

11. A composition comprising a dendrimer and at least one matrix molecule suitable for mass spectrometry.

12. The composition of claim 11, wherein the composition comprises a poly(ethylene glycol) (PEG) dendrimer or a poly(amidoamine) (PAMAM) dendrimer.

13. The composition of claim 11, wherein the least one matrix molecule comprises sinapinic acid (SA), alpha-cyano-4-hydroxycinnamicacid (CHCA), 2,5-dihydroxybenzoicacid (DHB), 3-hydroxypicolinic acid, 2′,4′,6′-trihydroxyacetophenone (THAP) 2-(4-hydroxyphenylazo)benzoic acid, succinic acid, anthranic acid, nicotinic acid, salicylamide, isovanillin, 3-aminoquinoline, 1-sioquinolinol, or Dithranol.

14. The composition of claim 11, further comprising at least one compound that is not a matrix molecule.

15. The composition of claim 14, where the at least one compound comprises a protein, polypeptide, oligopeptide, amino acid, monosaccharide, disaccharide, polysaccharide, nucleotide, oligonucleotide, polynucleotide, proteoglycan, glycoprotein, lipid, lipoprotein, natural polymer, or a soluble synthetic polymer.

16. The composition of claim 11 further comprising a solid support, wherein the composition is disposed on the solid support.

17. The composition of claim 16, wherein the solid support is a sample platform suitable for mass spectrometry.

18. The composition of claim 11, further comprising a stabilizer, additive or preservative.

19. The composition of claim 18, wherein the stabilizer, additive or preservative is an anticoagulant, a procoagulant, an antimicrobial agent, an antioxidant or a protease inhibitor.

20. The composition of claim 19, wherein the stabilizer is a protease inhibitor.

21. The composition of claim 19, wherein the anticoagulant is EDTA, heparin, or citrate.

22. The composition of claim 11, further comprising a labile internal standard.

23. The composition of claim 22, wherein the labile internal standard is sensitive to heat or a protease.

24. An arrangement comprising a composition and a specimen collection container, wherein the composition comprises a dendrimer and the specimen collection container comprises an internal chamber that is partially evacuated.

25. The arrangement of claim 24, wherein the composition further comprises a labile internal standard.

26. The arrangement of claim 25, wherein the labile internal standard is sensitive to heat or a protease.

27. The arrangement of claim 24, wherein the composition further comprises a stabilizer, additive or preservative.

28. The arrangement of claim 27, wherein the stabilizer, additive or preservative is an anticoagulant, a procoagulant, an antimicrobial agent, an antioxidant and a protease inhibitor.

29. The arrangement of claim 28, wherein the anticoagulant is EDTA, heparin, or citrate.

30. The arrangement of claim 24, wherein the dendrimer is a poly(ethylene glycol) (PEG) dendrimer or a poly(amidoamine) (PAMAM) dendrimer.

31. A method of aligning mass spectra, comprising obtaining a first mass spectrum from a first sample comprising a dendrimer, obtaining a second mass spectrum from a second sample comprising a dendrimer, and aligning the first spectra and second spectra by reference to an m/Z value or a time-of-flight of the dendrimer in the first and second mass spectra.

32. The method of claim 31, wherein the dendrimer is a poly(ethylene glycol) (PEG) dendrimer or a poly(amidoamine) (PAMAM) dendrimer.

33. The method of claim 31, wherein the mass spectrometry of step b) comprises laser desorption/ionization-type spectrophotometry.

34. The method of claim 33, wherein the laser desorption/ionization-type spectrophotometry comprises matrix-assisted laser desorption/ionization (MALDI), surface enhanced laser desorption/ionization (SELDI), MALDI-time-of-flight (MALDI-TOF), SELDI-TOF, MALDI-TOF-mass spectrometry (MS), SELDI-TOF-MS, SELDI-Q-TOF, SELDI-MS/MS, or any combination thereof.

35. The method of claim 33, wherein the mass spectrometry comprises electrospray ionization.
